(19) United States
(12) Patent Application Publication
(10) Pub. No.: US 2008/0115783 A1
(43) Pub. Date: May 22, 2008

(54) Laryngeal Mask Airway Device

(76) Inventor: Archibald I.J. Brain, Les Bons Villers (BE)

Correspondence Address: WILMERHALE/BOSTON, 60 STATE STREET, BOSTON, MA, 02109, US

(21) Appl. No.: 11/578,689

(22) PCT Filed: Apr. 14, 2005
(86) PCT No.: PCT/EP05/04149

§ 371 Date: Jan. 28, 2008

(30) Foreign Application Priority Data
Apr. 16, 2004 (GB) 0408548.6

(51) Int. Cl. A61B 1/267; A61M 16/00; A62B 9/00

(52) U.S. Cl. 128/200.26; 128/207.14; 600/120

(57) ABSTRACT

The invention relates to a laryngeal mask airway device (1) for insertion into a patient to provide an airway passage to the patient's glottic opening, the device comprising an airway tube(2), a mask (3) attached to the airway tube, the mask comprising a body (4) including a peripheral inflatable cuff (5), an outlet (6) and an inlet (7), the mask being attached to the airway tube via the inlet for gaseous communication between the tube and the outlet, there being at least one fibre-optic cable (23, 24) terminating adjacent the outlet for receiving, in use, an image of the patient's glottic opening, and means (50) for viewing the image, the viewing means being disposed such that the viewing means remains in the user's field of view during manipulation of the device by the user to view the glottic opening.

The present invention relates to a laryngeal mask airway device, and more specifically to an intubating laryngeal mask airway device with a fibre optic assembly.

The laryngeal mask airway device is a well known device that is useful for establishing airways in unconscious patients. U.S. Pat. No. 4,509,514 is one of the many publications that describe laryngeal mask airway devices. Such devices have been in use for many years and offer an alternative to the older, even better known endotracheal tube. For at least seventy years, endotracheal tubes comprising a long slender tube with an inflatable balloon disposed at the tube's distal end have been used for establishing airways in unconscious patients. In operation, the endotracheal tube's distal end is inserted through the mouth of the patient, past the patient's trachea. Once so positioned, the balloon is inflated so as to form a seal with the interior lining of the trachea. After this seal is established, positive pressure may be applied to the tube's proximal end to ventilate the patient's lungs. Also, the seal between the balloon and the inner lining of the trachea protects the lungs from aspiration (e.g., the seal prevents material regurgitated from the stomach from being aspirated into the patient's lungs).

Although they have been enormously successful, endotracheal tubes suffer from several major disadvantages. The principal disadvantage of the endotracheal tube relates to the difficulty of properly inserting the tube. Inserting an endotracheal tube into a patient is a procedure that requires a high degree of skill. Also, even for skilled practitioners, insertion of an endotracheal tube is sometimes difficult or not possible. In many instances, the difficulty of inserting endotracheal tubes has tragically led to the death of a patient because it was not possible to establish an airway in the patient with sufficient rapidity. Also, inserting an endotracheal tube normally requires manipulation of the patient's head and neck and further requires the patient's jaw to be forcibly opened widely. These necessary manipulations make it difficult, or undesirable, to insert an endotracheal tube into a patient who may be suffering from a neck injury.

In contrast to the endotracheal tube, it is relatively easy to insert a laryngeal mask airway device into a patient and thereby establish an airway. Also, the laryngeal mask airway device is a “forgiving” device in that even if it is inserted improperly, it still tends to establish an airway. Accordingly, the laryngeal mask airway device is often thought of as a “life saving” device. Also, the laryngeal mask airway device may be inserted with only relatively minor manipulation of the patient's head, neck and jaw. Further, the laryngeal mask airway device provides ventilation of the patient's lungs without requiring contact with the sensitive inner lining of the trachea and the size of the airway established is typically significantly larger than the size of the airway established with an endotracheal tube. Also, the laryngeal mask airway device does not interfere with coughing to the same extent as endotracheal tubes. Largely due to these advantages, the laryngeal mask airway device has enjoyed increasing popularity in recent years.

U.S. Pat. Nos. 5,303,697 and 6,079,409 describe examples of prior art devices that may be referred to as “intubating laryngeal mask airway devices.” The intubating device is useful for facilitating insertion of an endotracheal tube. After an incubating laryngeal mask airway device has been located in the patient, the device can act as a guide for a subsequently inserted endotracheal tube. Use of the laryngeal mask airway device in this fashion facilitates what is commonly known as “blind insertion” of the endotracheal tube. Only minor movements of the patient's head, neck and jaw are required to insert the intubating laryngeal mask airway device, and once the device has been located in the patient, the endotracheal tube may be inserted with virtually no additional movements of the patient. This stands in contrast to the relatively large motions of the patient's head, neck and jaw that would be required if the endotracheal tube were inserted without the assistance of the intubating laryngeal mask airway device. Furthermore, these devices permit single-handed insertion from any user position without moving the head and neck of the patient from a neutral position, and can also be put in place without inserting fingers in the patient's mouth. Finally, it is believed that they are unique in being devices which are airway devices in their own right, enabling ventilatory control and patient oxygenation to be continuous during intubation attempts, thereby lessening the likelihood of desaturation.

In Applicant's own WO 95/33506 there is described an intubating laryngeal mask airway device with fibre-optic assembly. Although the device shown in that application has proved to be extremely successful in use, a number of operational difficulties have been encountered and it is an object of the present invention to meet those difficulties. In particular, one difficulty which has arisen results from the fact that it is often difficult for a user to simultaneously insert the endotracheal tube accurately and quickly whilst maintaining control over the viewing apparatus of the fibre-optic device.

According to the invention there is provided a laryngeal mask airway device for insertion into a patient to provide an airway passage to the patient's glottic opening, the device comprising an airway tube, a mask attached to the airway tube, the mask comprising a body including a peripheral inflatable cuff, an outlet and an inlet, the mask being attached to the airway tube via the inlet for gaseous communication between the tube and the outlet, there being at least one fibre-optic cable terminating adjacent the outlet for receiving, in use, an image of the patient's glottic opening, and means for viewing the image, the viewing means being disposed such that the viewing means remains in the user's field of view during manipulation of the device by the user to view the glottic opening.

The invention thus enables a user to maintain hand-eye coordination because, while manipulating the device, or an endotracheal tube inserted through the device, there is no need for the user to look in a different direction to the direction of the anatomy and the users' hands.

It is preferred that the viewing means is disposed such that it substantially overlies the larynx of a patient when the device is in place, in use. This means that the user sees the larynx in the same position as the actual anatomy.

The viewing means may be releasably disposed, and may preferably be disposed upon the airway tube, again only preferably, by mounting means of the airway tube.

The mounting means may be disposed substantially to avoid fouling on the patient's anatomy.

It is preferred that the mounting means is movable between a mounting position for mounting of viewing apparatus, and a stowed position, and a particularly preferred arrangement has the mounting means pivotally movable between the said positions. The mounting means may preferably comprise a hingeable peg.

It is preferred that the device includes light emitting fibre-optic cables and light receiving fibre-optic cables. The light emitting fibre-optic cables and the light receiving fibre-optic cables may be separate, and the separate cables may run from the body separately on opposite sides of the body.

As an alternative, the separate cables may run from the body together on the same side.

It is preferred that each cable runs in a lumen formed in the material of the body and it is further preferred that each said lumen includes a Teflon lining to protect the material of the body and the cables and allow for easy insertion of the fibre-optic cables.

In one particularly preferred form of the invention, the separate cables may converge at a side of the mask remote from the outlet and it is preferred that the point of convergence is spaced from the mask end of the airway tube so that damage to the cables caused by contact with the end of the airway tube is avoided. The cables will preferably pass from the point of convergence through the mask body to the outlet side thereof.

The viewing means may comprise any suitable viewing device for use with fibre optics, as are well known in the art. For example, the viewing device may comprise an LCD screen or an optical device such as an eye-piece.

According to a second aspect of the invention there is provided a laryngeal mask airway device for insertion into a patient to provide an airway passage to the patient's glottic opening, the device comprising an airway tube, and a mask attached to the airway tube, the mask comprising a body including a peripheral inflatable cuff, an outlet and an inlet, the mask being attached to the airway tube via the inlet for gaseous communication between the tube and the outlet, there being at least one fibre-optic cable terminating adjacent the outlet for receiving, in use, an image of the patient's glottic opening, and means for mounting viewing apparatus to said device for viewing said image, wherein the mounting means is disposed to facilitate connection of viewing means such that the viewing means remains in the user's field of view during manipulation of the device by the user to view the glottic opening.

According to a third aspect of the invention there is provided a method of tracheal intubation, comprising the use of a device as defined hereinabove.

The invention will further be described by way of example with reference to the accompanying drawings in which:

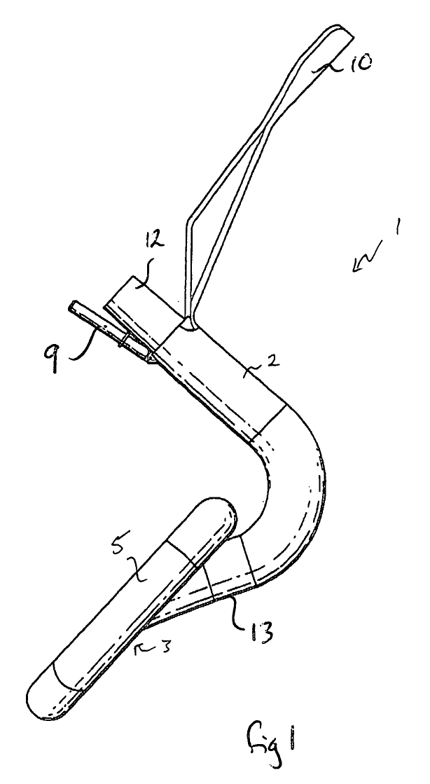

FIG. 1 is a side view of a first embodiment of device according to the invention in a first position;

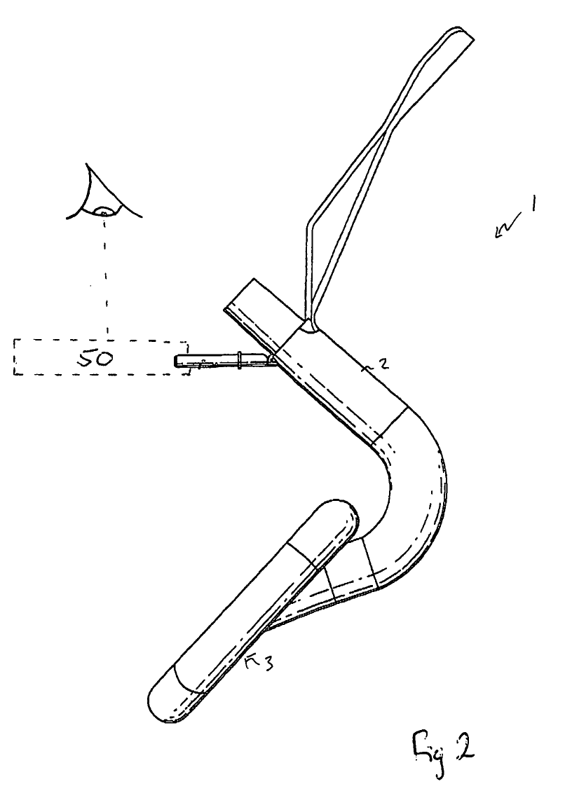

FIG. 2 is a side view of the device of FIG. 1 in a second position;

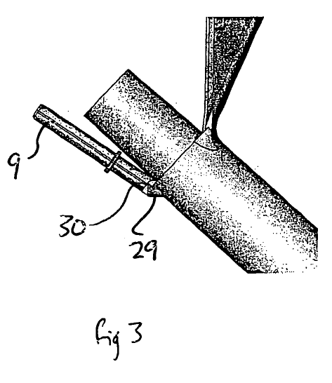

FIG. 3 is an enlarged view of part of FIG. 2;

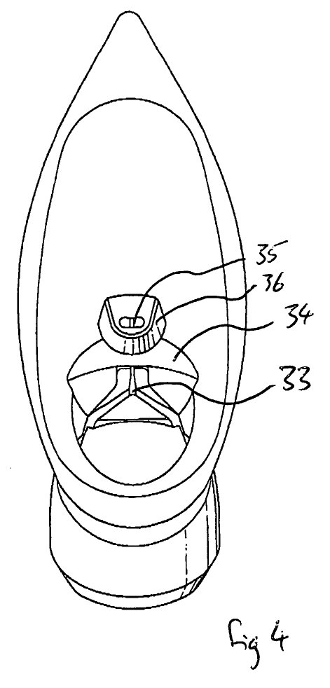

FIG. 4 is a front view of a part of a mask for use in a second embodiment of device according to the invention;

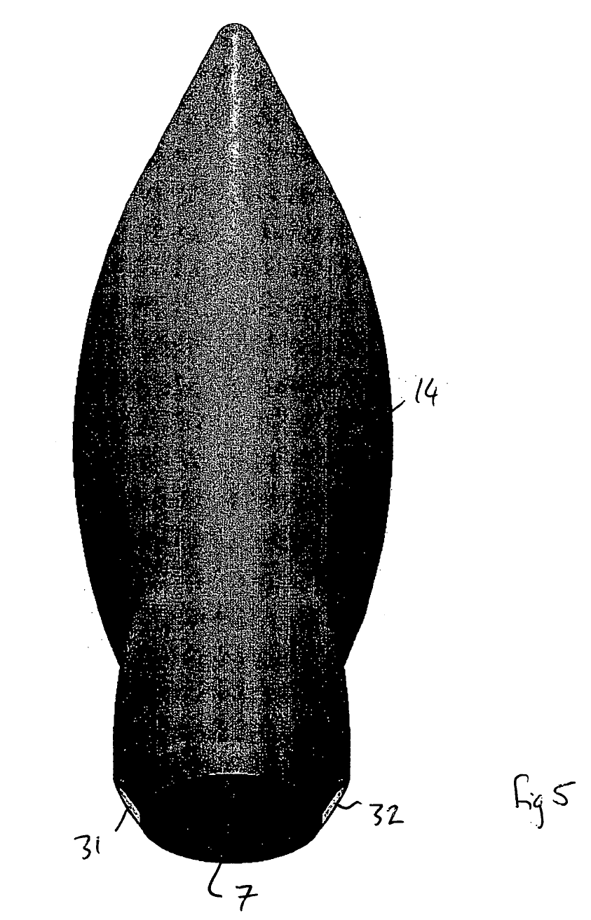

FIG. 5 is a back view of the part illustrated in FIG. 4;

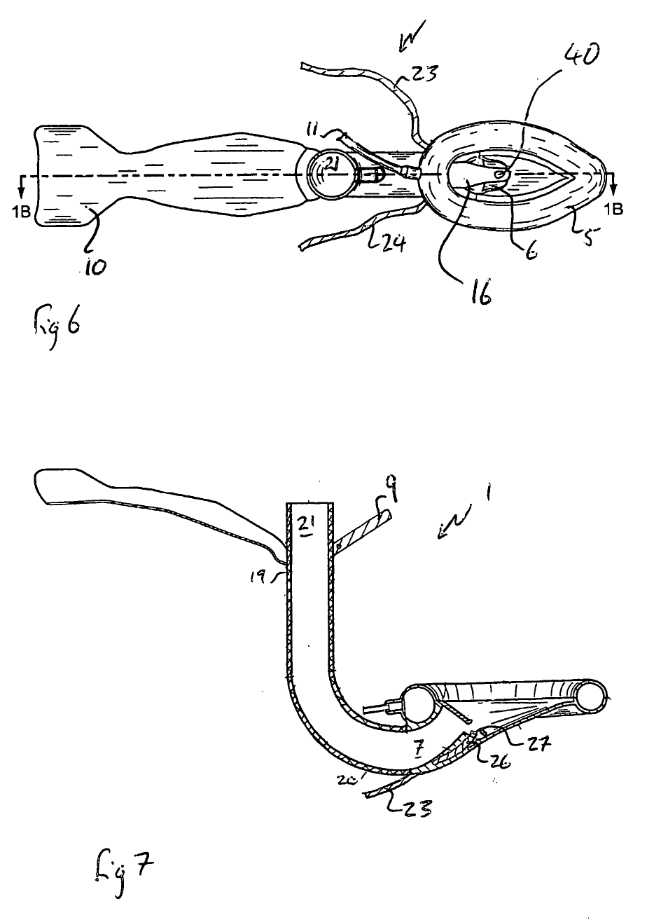

FIG. 6 is a plan view of the device of FIGS. 1 to 3;

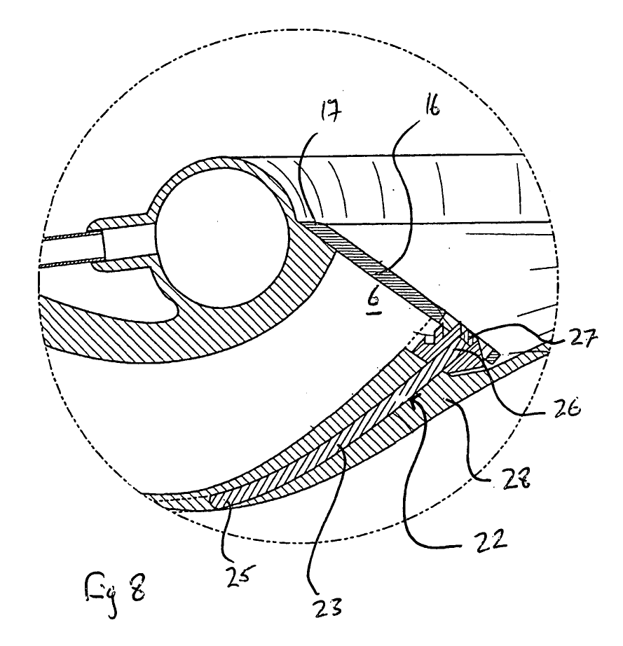

FIG. 7 is a sectional view of the device shown in FIG. 6; and

FIG. 8 is an enlarged view of a part of the device of FIGS. 6 and 7.

Referring to the drawings there is illustrated a laryngeal mask airway device 1 for insertion into a patient to provide an airway passage to the patient's glottic opening, the device comprising an airway tube 2, a mask 3 attached to the airway tube 2, the mask comprising a body 4 including a peripheral inflatable cuff 5, an outlet 6 and an inlet 7, the mask being attached to the airway tube via the inlet for gaseous communication between the tube 2 and the outlet 6, therebeing at least one fibre-optic cable 8 terminating adjacent the outlet 6 for receiving, in use an image of the patient's glottic opening, and means 50 for viewing the image, the viewing means 50 being disposed such that the image on the viewing means remains in the user's field of view during manipulation of the device by the user to view the glottic opening.

Device 1 includes a rigid airway tube 2, a silicone mask 3, a rigid handle 10 and an inflation line 11. The handle 10 is attached to the airway tube 2 near a proximal end 12 of the tube. Mask 3 is attached to airway tube 2 at a distal end 13 of the tube. Mask 3 includes a dome shaped silicone backplate 14 and an inflatable cuff 5. Mask 3 also includes an epiglottis elevator bar 16 (FIG. 6). Such epiglottis elevator bars 16 are known in the art, as illustrated for example in the Applicant's own WO 97/12641 (PCT/GB96/02426). One end 17 of bar 16 is attached to the mask 3. The other end 18 of the bar 16 is “free floating”, or not attached to any other portion of the device. As shown in FIGS. 1 to 3, the airway tube 2 defines a curved region that extends from a proximal ray 19 to a distal ray 20. As shown in FIGS. 5 and 6, the backplate 14 defines a ramp 28.

As shown best in FIG. 7, airway tube 2 defines a central airway passage 21. Central airway passage 21 extends from the proximal end 12 to the distal end 13 of the tube. When device 1 is inserted into a patient and the cuff 5 is inflated, the cuff 5 forms a seal around the patient's glottic opening and the airway passage 21 communicates with the patient's lungs. When the device 1 is inserted into a patient, the handle 10 and the proximal end 12 of the airway tube 2 remain outside of the patient's mouth, and the device 1 provides a sealed airway passage that extends from the proximal end 12 to the airway tube 2, through passage 21, to the patient's glottic opening.

As shown for example in FIGS. 6 and 7, device 1 includes a fibre-optic system 22. Fibreoptic system 22 includes two bundles of optical fibres 23, 24 that extend from a proximal end 25 to a distal end 26. In this embodiment the device 1 has two bundles of optical fibres because one bundle 23 is a viewing bundle, through which a view is obtained, and the other bundle 24 is an illumination bundle, through which light is passed to illuminate the subject under scrutiny. It is possible to provide a device which includes a single viewing bundle, with no illumination bundle. The viewing bundle has a lens 27 mounted at its distal end. When device 1 is inserted into a patient's mouth, the proximal ends 25 of bundles 23, 24 remain outside of the patient's mouth and may be connected to standard viewing devices (e.g., screens or eyepieces).

Although the curve of the airway tube 2 and the shape of the back plate 14 generally facilitate blind insertion of an endotracheal tube (not shown), the fibre-optic system 22 advantageously provides a view of the patient's anatomy that is aligned with the distal end of device 1. This enables alignment between the distal end of the device and the patient's glottic opening to be adjusted before attempting to insert an endotracheal tube through the device 1. If the distal end of the device is not perfectly aligned with the patient's glottic opening, as shown by the fibre-optic view obtained, the handle 10 may be used to make minor adjustments in the position of device 1 to thereby facilitate subsequent insertion of an endotracheal tube. This stands in contrast with prior art devices in which the glottic opening is sought and identified by means of an expensive mechanism built into the fibre-optic cable itself which allows its distal tip to be flexed in a single plane.

As shown in FIGS. 1 to 3, the device 1 includes means 9 for mounting viewing apparatus 50 to the device 1. The viewing apparatus 50 is mounted in a position where it remains in the user's field of view whilst the user manipulates the device 1 into position in a patient, and further whilst the user inserts an endotracheal tube. Thus, while manipulating either the device 1 or an endotracheal tube, the user is not required to look in a different direction to the direction of the actual anatomy. As a result, arm-brain coordination is preserved. The mounting means 9 in this embodiment of the invention comprises a peg of generally oval cross-section which is attached to the airway tube 2. The peg is formed from a rigid material such as steel and is attached to the tube 2 towards it proximal end 12, at about the same distance therefrom as the handle 10 but on the opposite side. Its position on the airway tube 2 however is mainly dictated by factors such as the requirement for avoiding fouling on a part of the patient's anatomy, whilst still being easily accessible for attachment and use of viewing apparatus. The peg is attached to the airway tube 2 in this embodiment so as to be pivotable between a position in which it is nearly flush with the airway tube 2, or “stowed”, and a position in which it stands proud therefrom at an angle of about 45° thereto. Attachment may be by any convenient means known to the skilled worker. In this embodiment a proximal end of the peg has a through hole, and the airway tube 2 is provided with two parallel upstands 29 (FIG. 3) both of which also have through holes which are in alignment. A pin 30 is passed through the holes in upstands 29 and the hole in the peg, thereby pivotably attaching the peg. As will be appreciated, the peg need not be pivotably attached, but may instead be fixedly attached in the upright position. Pivotable attachment is advantageous because it allows the peg to be stowed when it is not needed, and because it allows for the adjustment of the viewing angle of viewing apparatus. This can be important because once the device 1 is in place in the patient it is undesirable to have to move it because such movement may disturb its correct placement. A pivotable attachment also allows for adjustment of the viewing angle of viewing apparatus 50 such as an LCD screen which can be difficult to see from some viewing angles and in some light conditions. The peg 2 is constructed to allow fast and easy, but secure attachment of viewing apparatus.

Referring now to the embodiment illustrated in FIGS. 4 and 5, there is illustrated a part of a device 1, the part being the back plate 14. The other structures of this embodiment are all as shown for the embodiment of FIGS. 1 to 3 and 6 to 8 which have been omitted for clarity of view and description. As mentioned above, fibre-optic system 22 includes two bundles of optical fibres 23, 24 which extend to a distal end 26. The fibre bundles 23, 24 run into lumens 31, 32 (FIG. 5) which are moulded into the material of the mask. The bundles are not shown in FIG. 5 for clarity. The inside wall of each lumen 31, 32 is provided with a Teflon coating, which makes insertion of the fibre-optic cables easier and affords them some protection from damage. In this embodiment, the entrances to the lumens are located on the mask body 4 one on each side of the inlet 7, so that when the device 1 is inserted into a patient the fibre-optic cables are not damaged by contact with the patient's teeth to the upper and lower, in use, surfaces of the device 1, which often occurs. As will be appreciated, the lumens 31, 32 could also be placed together on one side of the inlet 7.

Referring to FIG. 4, from which the epiglottis elevator bar 16, the fibre-optics and the cuff have been omitted for clarity, from their entrances by the inlet 7, the lumens 31, 32 extend through into the material of the back plate 14 converging at a junction point 33 and passing through the material to emerge therefrom adjacent the outlet 6, at an enlarged, or thickened area of material 34. From the point of convergence 33 the two lumens 31, 32, become one 35 which passes through this enlarged or thickened area of material 34 to a nose 36, where the fibres emerge, terminating in lenses for illuminating the subject to be viewed, and receiving light reflected therefrom. As shown in FIG. 6, in all embodiments the epiglottic elevator bar 16 includes an aperture 40 so that the line of view from the fibres to the subject is not obscured when the bar is in the lowered position.

1. A laryngeal mask airway device for insertion into a patient to provide an airway passage to the patient's glottic opening, the device comprising an airway tube, a mask attached to the airway tube, the mask comprising a body including a peripheral inflatable cuff, an outlet and an inlet, the mask being attached to the airway tube via the inlet for gaseous communication between the tube and the outlet, there being at least one fibre-optic cable terminating adjacent the outlet for receiving, in use, an image of the patient's glottic opening, and means for viewing the image, the viewing means being disposed such that the viewing means remains in the user's field of view during manipulation of the device by the user to view the glottic opening.

2. A device according to claim 1, wherein the viewing means is disposed such that it substantially overlies the larynx of a patient when the device is in use.

3. A device according to claim 1 or claim 2, wherein the viewing means is releasably disposed.

4. A device according to any one of claims 1 to 3, wherein the viewing means is disposed upon the airway tube of the device.

5. A device according to claim 4, wherein the viewing means is disposed upon the airway tube via mounting means of the airway tube.

6. A device according to any preceding claim, wherein the mounting means is disposed to substantially avoid fouling on the patient's anatomy.

7. A device according to any preceding claim 2, wherein the airway tube comprises a rigid material.

8. A device according to any preceding claim, wherein the mounting means is moveable between a mounting position for mounting of viewing apparatus, and a stowed position.

9. A device according to claim 8, wherein the mounting means is pivotably movable between the said positions.

10. A device according to claim 9, wherein the mounting means comprises a hingeable peg.

11. A device according to any preceding claim, including light emitting fibre-optic cables and light receiving fibre-optic cables.

12. A device according to claim 11, wherein the light emitting fibre-optic cables and the light receiving fibre-optic cables are separate.

13. A device according to claim 12, wherein the said separate cable run from the body separately on opposite sides of the body.

14. A device according to claim 12, wherein the said separate cables run from the body together on the same side.

15. A device according to claim 13 or claim 14, wherein each cable runs in a lumen formed in the material of the body.

16. A device according to any of claims 12 to 15, wherein the separate cables converge at a side of the mask remote from the outlet.

17. A device according to claim 16, wherein the point of convergence is spaced from the mask-end of the airway tube.

18. A device according to claim 16 or claim 17, wherein the cables run from the point of convergence through the mask to the outlet side thereof.

19. A device according to claim 18, when dependent on any of claims 16 to 17, wherein each lumen includes a Teflon lining.

20. A device according to any preceding claim, wherein the viewing means comprises an LCD device.

21. A laryngeal mask airway device for insertion into a patient to provide an airway passage to the patient's glottic opening, the device comprising an airway tube, and a mask attached to the airway tube, the mask comprising a body including a peripheral inflatable cuff, an outlet and an inlet, the mask being attached to the airway tube via the inlet for gaseous communication between the tube and the outlet, there being at least one fibre-optic cable terminating adjacent the outlet for receiving, in use, an image of the patient's glottic opening, and means for mounting viewing apparatus to said device for viewing said image, wherein the mounting means is disposed to facilitate connection of viewing means such that the viewing means remains in the user's field of view during manipulation of the device by the user to view the glottic opening.

22. A method for tracheal intubation, comprising the use of a device as claimed in any of claims 1 to 21.
